(19) United States
(12) Patent Application Publication
(10) Pub. No.: US 2025/0155375 A1
(43) Pub. Date: May 15, 2025

(54) METHOD AND SYSTEM FOR DETERMINING AT LEAST ONE OF THE CHARACTER, COMPOSITION AND REACTIVITY OF DISSOLVED ORGANIC MATTER IN WATER

(71) Applicant: VALYS AB, Göteborg (SE)

(72) Inventor: Kathleen MURPHY, Göteborg (SE)

(21) Appl. No.: 18/839,098

(22) PCT Filed: Feb. 21, 2023
(86) PCT No.: PCT/SE2023/050155

§ 371 Date: Aug. 16, 2024

(30) Foreign Application Priority Data
Feb. 23, 2022 (SE) 2250244-7

(51) Int. Cl. G01N 21/64; G01N 1/40; G01N 33/18

(52) U.S. Cl. CPC G01N 21/645; G01N 1/4077; G01N 21/6486; G01N 33/1826; G01N 2001/4088

(57) ABSTRACT

The present disclosure relates to a system (1) for determining at least one of the character, composition and reactivity of dissolved organic matter, DOM in water comprising: an ultraviolet, UV light source (2) configured to excite a water under examination (3) with a light beam (2′) having an excitation wavelength in the range of 250-500 nm. Further, the system comprises a first detector device (4) and a second detector device (5), wherein the system (1) further comprises control circuitry (6) configured to predict at least one of the composition, character and reactivity of DOM in the water under examination (3) based on said first and second fluorescence intensity.

### TECHNICAL FIELD

The present invention relates to a method and a system for determining the character, composition or reactivity of dissolved organic matter in water.

### BACKGROUND ART

All aquatic environments contain a diverse mixture of organic chemicals that are produced by living organisms (e.g., via primary productivity by bacteria and algae) or that represent the decomposition products remaining after organisms die. These chemicals are collectively referred to as natural organic matter (NOM). A large fraction of NOM compounds are in dissolved form (DOM) and these are often quantified by measuring dissolved organic carbon (DOC), since carbon is a major element in DOM. Knowing the composition of DOM in water is useful for determining what types of processes will remove it most effectively during water treatment. More broadly in environmental applications, DOM composition affects its reactivity and hence the type and rate of transformation processes that occur when DOM is transported through groundwater, lakes, and rivers and out to the oceans. This is useful for predicting the fate of carbon in aquatic systems and for developing carbon budgets.

A way to obtain indications of the quality of DOM in a water sample of water is to measure by fluorescence spectroscopy, which is cheaper and more rapid than conventional offline methods based on chromatography, and is more sensitive than absorbance spectroscopy. Fluorescence is based on the principle that a subset of dissolved organic matter compounds re-emit some of the light that they absorb according to characteristic absorption and emission spectra that depend on their chemical structure and properties. Fluorescence spectroscopy thus measures signals emitted by dissolved chemicals, including DOM, after they have absorbed light at particular energy levels (corresponding to particular wavelengths) and then re-emitted some of this light at lower energy levels (i.e. at longer wavelengths). In a water sample, the combination of absorbance (excitation) and fluorescence spectra can be used to get an indication of the relative amounts of different types of dissolved organic compounds and the overall reactivity of the DOM.

This is important because treatment success depends on the nature and reactivity of DOM in water as well as its quantity. It is particularly useful to determine DOM reactivity in real time in order to quickly adapt treatment conditions, including chemical doses, flow rates and/or contact times, in response to changes in incoming water quality. Efficient systems are therefore needed to measure DOM reactivity in real time especially under conditions of fluctuating raw water quality, thus making it possible to continuously optimise treatment conditions. In today's water treatment plants, there are no efficient systems directed to measuring the character, composition and reactivity of dissolved organic matter in water. Instead, the known methods and systems are primarily directed to measure the overall quantity of organic matter present according to bulk parameters such as total organic carbon (TOC) or UV-254 nm absorbance (A254). Systems for measuring the character, composition and reactivity of dissolved organic matter in water typically require that samples are sent to external analytical laboratories, making analyses expensive to implement and extending the time needed to detect changes in water quality. Additionally, the known methods and systems used at external laboratories commonly have high detection limits and are subject to a range of interferences that frequently result in inaccurate or imprecise measurements.

Additionally, although online systems exist for detecting when water is contaminated by polyaromatic hydrocarbons (for example, petroleum), existing systems produce data that can be difficult to interpret or use because they cannot distinguish between signals due to hydrocarbons present at low concentrations versus signals due to natural organic matter present at high concentrations. Thus, based on the above, there is a need for improved methods and systems for determining the composition of water and the character and reactivity of dissolved organic matter, more specifically there is a need for such methods and systems that are improved in terms of analysis time, precision, sensitivity and cost-efficiency.

### SUMMARY

It is therefore an object of the present disclosure to provide a system and a method to mitigate, alleviate or eliminate one or more of the above-identified deficiencies and disadvantages.

This object is achieved by means of a method and a system as defined in the appended claims. The present disclosure provides a system for determining at least one of the character, composition and reactivity of dissolved organic matter, DOM in water. The system comprising an ultraviolet, UV light source configured to excite a sample of water under examination with a light beam having an excitation wavelength in the range of 250-500 nm. A first detector device configured to determine a first fluorescence intensity emitted from said sample of water, the first detector device being arranged to measure at a first emitted wavelength being in the range of 375-405 nm. Moreover, the system comprises a second detector device configured to determine a second fluorescence intensity emitted from said sample of water, the second detector device being arranged to measure at a second emitted wavelength being in the range of 490-580 nm. Furthermore, the system further comprises control circuitry configured to predict/determine at least one of the composition, character and reactivity of DOM in the sample of water under examination based on said first and second fluorescence intensity. In some aspects herein, the first and the second detector devices may be a common detector device, e.g. a spectrometer. The excitation wavelength in the range of 250-500 allows for exciting the long-emission, aromatic fraction of NOM, which is feasible to do at a wide range of excitation wavelengths. It may be preferable to excite this fraction using different wavelengths to the non-aromatic fraction if the instrument contains a light source that includes excitation wavelengths in the range of 250-305 nm or 335-500 nm, which is outside the optimal range of excitation wavelengths for the less aromatic NOM fraction that is measured using the first detector.

It should be noted that the water may be a sample of water held in e.g. a container. Accordingly, the system may further comprise a container for holding a sample of water under examination. Moreover, the water under examination may be water in e.g. a lake or ocean. The system of the present disclosure may therefore be portable.

An advantage of the system of the present disclosure is that dissolved organic matter in water can be targeted in an efficient manner based on the ranges of the wavelengths of the present disclosure. Accordingly, the character, composition and reactivity of DOM can be predicted so to allow the water to be treated accordingly after said prediction. The system of the present disclosure may utilize two different emission wavelengths for a single excitation wavelength, to target different DOM constituents allowing for an improved but cost-efficient prediction. Additionally, this design ensures that if external factors (e.g. change in the water sample's optical density or temperature) or internal factors (e.g. lamp deterioration, power fluctuations) cause fluctuations in the amount of light absorbed by the sample, this affects both emission detectors equally and therefore does not affect the ratio of measurements by the two detectors. The detectors combined with the UV light source allow for at least two

DOM components to be targeted, wherein one of said DOM components may be utilized in the method to track the aromatic fraction of DOM that is reactive and easy to treat, while the other DOM component may track a recalcitrant fraction that is difficult to treat.

Further, the system of the present disclosure provides the advantage of sensitive, real-time information on water composition and reactivity allowing drinking water treatment to be continuously adjusted and optimized. Thereby leading to better treatment outcomes at lower operational cost and with lower environmental impacts. Thus it may provide benefits for water treatment producers such as improved prediction of disinfection byproduct formation potential, improved prediction of reversible and irreversible membrane fouling potential, assessment of adsorption capacity in granular activated carbon filters, detection of petroleum contamination and of contamination by other anthropogenic pollutants, improved prediction of optimal chemical dose for coagulation and flocculation enabling better automated dosing control systems.

The excitation wavelength may be in the range of 305-335, preferably 310-330 nm, more preferably 313-325 nm. Moreover, the first emitting wavelength may be in the range of 380-400 nm, preferably, 385-395 nm. Moreover, the second emitted wavelength is in the range of 500-570 nm, preferably 510-540 nm.

An advantage of the abovementioned ranges, specifically the preferred ranges is that they allow for an even further improved prediction as the specific excitation and emission maxima of DOM components in the water are more efficiently targeted by minimising the potential for interferences by non-target signals such as contaminants or light scattering. Thus the method can selectively be more optimized by narrowing the ranges accordingly.

The control circuitry may be configured to predict based on a ratio of the first fluorescence intensity relative the second fluorescence intensity. The ratio derived from the water under examination may be (by the control circuitry) compared with pre-determined ratios from other samples having known properties (stored in the system) so to predict the composition, character and reactivity of DOM in the water under examination. Thus, the method provides the benefit of a rapid and efficient prediction of the composition, character and reactivity of DOM in the sample of water under examination based on a ratio of first and second fluorescence intensity. Additionally, the method is highly sensitive and precise in comparison to known methods and systems for predicting the composition, character and reactivity of DOM that are based on ratioing measurements that were collected using two separate instruments.

The system may comprise a third detector device configured to determine at a third fluorescence intensity emitted from said water under examination. The third detector device being arranged to measure at a third emitted wavelength being in the range of 410-460 nm. Further, the control circuitry may be configured to further base the prediction on said third fluorescence intensity.

An advantage of this is that the peak fluorescence emission intensity of the water under examination may be estimated based on the wavelength range measured by the third detector. Consequently, the prediction can be improved by incorporating an additional parameter that provides information about the overall quantity of fluorescent DOM in said water sample—which in combination with the two detectors that track DOM quality may provide a more accurate and reliable prediction.

The system may further comprise a first ultraviolet detector device configured to monitor an intensity of said ultraviolet light source, and a second ultraviolet detector device configured to measure an amount of light transmitted through said water under examination. The control circuitry may be configured to apply a correction factor to said prediction, said correction factor being based on the measured amount of light transmitted by said water under examination and the monitored intensity of said ultraviolet light source. In other words, the ultraviolet detectors may measure how much light is absorbed by the water under examination. The measurements by the first and second ultraviolet detector may further be used to derive the optical density of the water under examination so as to develop said correction factor which can be applied to the prediction to account for e.g. internal fluorescence quenching also known as inner filter effects. The first and the second detector device may within the scope of the present disclosure be a common device and is not limited to two separate units.

Further, it should be noted that the present disclosure is not limited to a single UV light source. In some aspects herein the UV light source is a first light source having an excitation wavelength of 305-335 nm, wherein the system further comprises a second UV light source configured to excite the water under examination with another light beam, the another light beam having an excitation wavelength in the range of 250-500 nm, wherein the first detector is arranged to detect fluorescence emissions due to the first light source and the second detector is arranged to detect fluorescence emissions due to the second light source, i.e. each detector may be arranged to detect emissions from a separate UV light source. Further, in some aspects the term “UV light source” may refer to a UV light source arrangement comprising several light sources in accordance with the aforementioned first and the second UV light source. Alternatively, it could refer to a broad-band light source used in combination with excitation filters to achieve excitation wavelengths in accordance with the aforementioned first and the second UV light source.

Additionally, by combining the measurements from three fluorescence detectors with the measurements from the two UV transmission detectors, the control circuitry may be configured to either estimate the full fluorescence emission spectrum of the water, and to estimate the sample's apparent quantum yield (AQY) in combination with reference data measured using a standard reference material (e.g. quinine sulfate). Thus, the method may be more flexible and provide additional estimations in alternative ways. Since unpolluted natural waters have similar values of AQY, large deviations in AQY can be used to detect anomalies or failures of various kinds. For example an anomalously high AQY value could indicate that the sample was contaminated by hydrocarbons e.g. crude oil, whereas an anomalously low AQY value could indicate a failing light source or emission detector, or too high optical density causing insufficient light transmission through the sample. The method could also be used to estimate DOC concentration by dividing the value predicted by the method by sample absorbance at 254 nm excitation wavelength (A254). A254 may be either measured directly or estimated by extrapolation of the light transmission measurements measured by the first and second ultraviolet detector devices (which may be a common detector device).

The system may further comprise a pre-filter device configured to exclude particles equal to or greater than 0.2 μm from the water under examination prior to determining fluorescence intensity by means of the first and the second detector device. An advantage of such a pre-filter device excluding particles greater than 0.2 μm is that this maximises the amount of light reaching the sample and detectors by minimising the amount of light scattered off particles. This results in a more sensitive and accurate measurement.

There is also provided a method for determining the character, composition or reactivity of dissolved organic matter, DOM in water, the method comprising the steps of:
exciting a sample of water under examination with an excitation emission having a wavelength in the range of 250-500 nm;
determining a first fluorescence intensity emitted from said sample of water by a first detector device measuring at a first emitting wavelength being in the range of 380-400 nm;
determining a second fluorescence intensity emitted from said sample of water by a second detector device measuring at a second emitted wavelength being in the range of 490-580 nm
predicting at least one of the composition, character and reactivity of DOM of the water under examination based on said first and second fluorescence intensity.

It should be noted that the method may be performed according to any aspect of the present disclosure, e.g. the ranges may be adapted in accordance with the preferred ranges.

Moreover, the step of prediction may be based on a ratio of the first fluorescence intensity relative the second fluorescence intensity.

The method may further comprise the step of determining a third fluorescence intensity emitted from said sample of water by a third detector device measuring at a third emitted wavelength being in the range of 410-460 nm so that the step of prediction is further based on said third fluorescence intensity.

Thus, in some aspects of the present disclosure when the method utilizes three detectors, the measurements by the three fluorescence detectors may further be used in combination to detect various anomalies.

The method may further comprise the steps of
monitoring, by a first ultraviolet detector device, an intensity of said ultraviolet light source;
determining, by a second ultraviolet detector device, an amount of light transmitted through said water under examination. Further, the method may also comprise the step of, after the step of predicting:
applying a correction factor to said prediction, said correction factor being based on the measured amount of light transmitted through said water under examination and the monitored intensity of said ultraviolet light source.

The method may further comprise the step of: excluding particles equal to or greater than 0.2 μm from the water under examination prior to determining fluorescence intensity by means of the first and the second detector device. The exclusion may be performed by a filtration device.

There is further provided a computer-readable storage medium storing one or more programs configured to be executed by one or more control circuitry of a system, the one or more programs including instructions for performing the method of any aspect of the present disclosure.

### BRIEF DESCRIPTION OF THE DRAWINGS

In the following, the disclosure will be described in a non-limiting way and in more detail with reference to exemplary embodiments illustrated in the enclosed drawings, in which:

FIG. 1 schematically illustrates a system 1 for determining at least one of the character, composition and reactivity of DOM in water in accordance with some embodiments of the present disclosure;

FIG. 2 schematically illustrates a system 1 for determining at least one of the character, composition and reactivity of DOM in water in accordance with some embodiments of the present disclosure;

FIG. 3 illustrates a flowchart of a method 100 for for determining at least one of the character, composition and reactivity of DOM in water in accordance with some embodiments of the present disclosure;

FIGS. 4A-4B illustrates graphs showing the location of detector measuring windows in relation to DOM components that are targeted by the disclosed method;

FIGS. 5A-5D illustrates graphs showing the location of measurement windows belonging to the first and second detector devices in relation to non-target DOM components:

FIGS. 6A-6C illustrates graphs showing emission scans for several water to exemplify the relationship between DOM fluorescence, scatter peak positions, excitation wavelength, and emission wavelength;

FIG. 7 illustrates a graph showing how the positions of the three emission detector devices target the left, right and centre of the DOM fluorescence peak in a dataset comprised of filtered samples from pristine rivers in Alaska;

FIGS. 8A-8B illustrates graphs showing the suitability of the method 100 and system 1 of the present disclosure for predicting SUVA;

FIG. 9 illustrates graphs showing the suitability of the method 100 and system 1 of the present disclosure for predicting SUVA;

FIG. 10 illustrates a graph demonstrating test results disclosing the suitability of the method 100 and system 1 of the present disclosure for predicting SUVA;

FIG. 11 illustrates graphs showing the optimal measurement windows for predicting H_{LEA}/H_{SED} and SUVA using the method 100 and system 1 of the present disclosure applied to low turbidity samples from a dataset; and

FIG. 12 illustrates a graph showing that the method 100 and system 1 of the present disclosure for quantifying changes in DOM composition caused by adsorption onto granular activated carbon (GAC) filters.

FIG. 13 is a graph showing that the method 100 and system 1 of the present disclosure may be utilized to predict drinking water treatability at different stages of water treatment.

### DETAILED DESCRIPTION

In the following detailed description, some embodiments of the present disclosure will be described. However, it is to be understood that features of the different embodiments are exchangeable between the embodiments and may be combined in different ways, unless anything else is specifically indicated. Even though in the following description, numerous specific details are set forth to provide a more thorough understanding of the provided disclosure, it will be apparent to one skilled in the art that the embodiments in the present disclosure may be realized without these details. In other instances, well known constructions or functions are not described in detail, so as not to obscure the present disclosure.

In the following description of example embodiments, the same reference numerals denote the same or similar components.

The phrase “fluorescence intensity” may refer to an amount of light emitted.

The phrase “dissolved organic matter (DOM)” may refer to a mixture of organic molecules found in water which are made up of carbon, hydrogen, and oxygen as well as the heteroatoms nitrogen and which pass through a filter of nominal size in the range 0.22-0.7 μm.

The phrase “water under examination” may refer to e.g. a sample of water (e.g. fresh water) held in a container subjected to the method and system of the present disclosure. However, it may also be water in the environment.

The phrase “emitting wavelength” may refer to a wavelength of light that is detected following its emission by fluorophores in a sample that was excited by light having a shorter wavelength.

The phrase “excitation wavelength” may refer to a wavelength of light that is absorbed by a fluorophores in a sample and results in fluorescence emission at a longer wavelength.

The phrase “composition, character and reactivity of dissolved organic matter” may refer to the average aromatic content of DOM in a sample measured by methods that may include nuclear magnetic resonance (NMR) spectroscopy, specific ultraviolet absorbance (SUVA) measurement, or liquid chromatography with organic carbon detection (LC-OCD). It may also refer to a characteristic of DOM related to aromaticity that helps to predict its behaviour in aquatic systems when subjected to physical, chemical or biological processes, including predicting its treatability in water treatment plants at various stages of treatment.

The phrase “SUVA” may refer to specific ultraviolet absorbance calculated by dividing the ultraviolet absorbance (UVA) measurement of sample at 254 nm (UVA254) by the DOC concentration of the same sample and multiplying by 100 to give a value reported as L/mg-m. SUVA may provide a characterization of the reactivity of DOM in a water under examination and may be utilized for estimating disinfection by-product formation potential.

The phrase “DOM component” may refer to a fraction of DOM that has been reported to occur in aquatic samples and that has different chemical properties compared to other reported fractions. Excitation and emission spectra for a DOM component may have been measured directly or may have been estimated using a statistical technique for example using parallel factor analysis (PARAFAC).

The phrase “turbidity” may refer to the abundance of particles in a sample. In samples with higher turbidity there is a higher incidence of light scattering, which has a negative impact on the precision and accuracy of spectroscopic (fluorescence and absorbance) measurements.

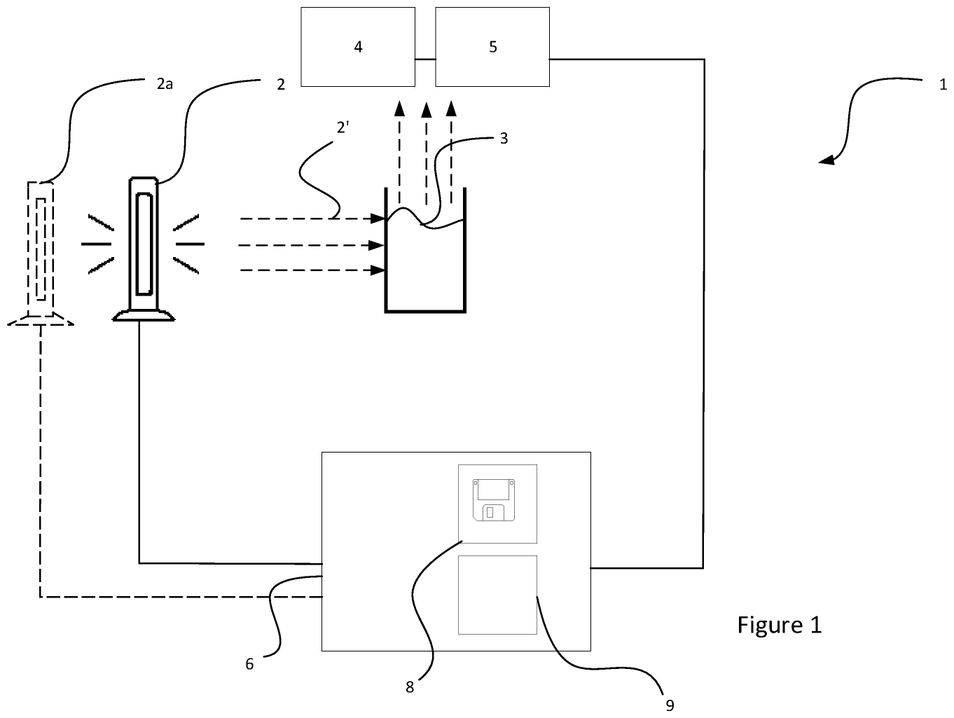

FIG. 1 illustrates a schematic view of a system 1 for determining at least one of the character, composition and reactivity of dissolved organic matter (DOM) in water, in accordance with the present disclosure. The system 1 comprises an ultraviolet, UV light source 2 configured to excite a water under examination 3 with a light beam 2′ having an excitation wavelength in the range of 250-500 nm. The excitation wavelength may be in the range of 310-330 nm, preferably 313-325 nm. The UV light source 2 may be a gas discharge lamp, a mercury lamp, a deuterium lamp, a metal vapour lamp, a laser, a light emission diode, or a plurality of light emission diodes. It should be noted that in some aspects herein the UV as illustrated in FIG. 1 the light source is a first light source 2 (as illustrated in FIG. 1) having an excitation wavelength of 305-335 nm, wherein the system 1 further comprises a second UV light source 2a configured to excite the water under examination with another light beam, the another light beam having an excitation wavelength in the range of 250-500 nm,. Accordingly, the first detector 4 is arranged to detect fluorescence emissions due to the first light source 2 and the second detector 5 is arranged to detect fluorescence emissions due to the second light source 2a, i.e. each detector 4, 5 may be arranged to detect emissions due from a separate UV light source.

Further, the system 1 comprises a first detector device 4 configured to determine a first fluorescence intensity emitted from said water under examination 3, the first detector device 4 being arranged to measure at a first emitting wavelength being in the range of 375-405 nm. The first emitting wavelength may be in the range of 380-400 nm, preferably, 385-395 nm.

Moreover, the system 1 comprises a second detector device 5 configured to determine a second fluorescence intensity emitted from said water under examination, the second detector device 5 being arranged to measure at a second emitting wavelength being in the range of 490-580 nm. The second emitted wavelength may be in the range of 500-570 nm, preferably 510-540 nm. Furthermore, the system 1 also comprises control circuitry 6 configured to predict at least one of the composition, character and reactivity of DOM in the water under examination 3 based on said first and second fluorescence intensity. Even though FIG. 1 illustrates a first and a second detector device 4, 5 the detector devices 4, 5 may in other aspects herein be a common detector device. Thus, the detector devices 4, 5 may be integrated into one device.

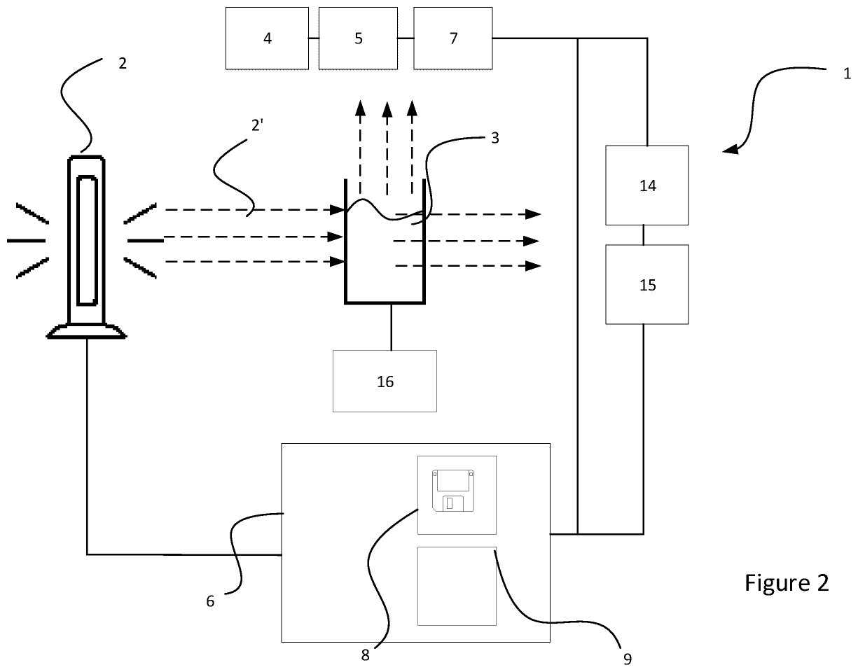

FIG. 2 illustrates an aspect of the system 1 further comprising a third detector device 7 configured to determine at a third fluorescence intensity emitted from said water under examination 3, the third detector device 7 being arranged to measure at a third emitted wavelength being in the range of 410-460 nm, said control circuitry 6 being configured to further base the prediction on said third fluorescence intensity. The third detector device 7 may also be a common device in accordance with aspects herein. Thus, in some aspects herein, the system 1 comprises one detector device that is configured to determine a first, a second and a third fluorescence intensity.

Moreover, the system 1 illustrates in FIG. 2 that it may further comprise a first ultraviolet detector device 14 configured to monitor an intensity of said ultraviolet light source 2 and a second ultraviolet detector device 15 configured to measure an amount of light transmitted through said water under examination 3. Further, the control circuitry 6 may be configured to apply a correction factor to said prediction, said correction factor being based on the measured amount of light transmitted through said water under examination 3 and the monitored intensity of said ultraviolet light sources 14, 15. The detectors may be optical detectors or electrical detectors.

FIG. 2 further illustrates that the system may further comprise a pre-filter device 16 configured to exclude particles equal to or greater than 0.2 μm from the water under examination 3 prior to determining fluorescence intensity by means of the first and the second (and optionally the third) detector device 4, 5.

Even though FIG. 2 illustrates the system having three detector devices 4, 5, 7, two ultraviolet detector devices 14, 15, the system 1 is not bound to such a specific configuration. Accordingly, the system 1 may have two ultraviolet detector devices combined with only two detector devices 4, 5. Moreover, the pre-filter device 16 may be incorporated into the system shown in FIG. 1.

It should be noted that FIGS. 1 and 2 are schematic figures and that the placement of the detectors 4, 5, 7, light source 2 and any other component (e.g. angles and distances) relative the water under examination 3 may vary in accordance with the knowledge of a skilled person utilizing the system 1. A person skilled in the art carrying out the present disclosure may recognize that fluorescence is detected at 90 degrees (or near to 90 degrees) to incident light direction and that absorbance is detected at 180 degrees (or near to 180 degrees). Further, each detector device 4, 5, 7 may comprise at least one band-pass filter, a high-pass filter, any combination thereof, or any other suitable type of filter for measuring at each specific wavelength.

The control circuitry 6 shown in FIGS. 1 and 2 may comprise one or more memory devices 8. The memory devices 8 may comprise any form of volatile or non-volatile computer readable memory including, without limitation, persistent storage, solid-state memory, remotely mounted memory, magnetic media, optical media, random access memory (RAM), read-only memory (ROM), mass storage media (for example, a hard disk), removable storage media (for example, a flash drive, a Compact Disk (CD) or a Digital Video Disk (DVD)), and/or any other volatile or non-volatile, non-transitory device readable and/or computer-executable memory devices that store information, data, and/or instructions that may be used by the control circuitry 6. Each memory device 8 may store any suitable instructions, data or information, including a computer program, software, an application including one or more of logic, rules, code, tables, etc. and/or other instructions capable of being executed by the detector devices 4, 5 and the control circuitry 6. Memory device 8 may be used to store any calculations/transactions/operations made by control circuitry 6 and the detectors 4, 5 and/or any data received via e.g. an input interfaces 9. In some embodiments, the control circuitry 6 and the detector devices 4, 5 are integrated. The control circuitry 6 may communicate/control and/or retrieve information from the light source 2 and the detector devices 4, 5 via wired or wireless connection, thereby monitoring the excitation wavelengths excited, and the emitting wavelengths measured at.

Each memory device 8 may also store data that can be retrieved, manipulated, created, or stored by the control circuitry 6 and the detector devices 4, 5. The data may include, for instance, local updates, parameters, learning models, user data. The data can be stored in one or more databases connected to the circuitry 6. The control circuitry 6 may store an algorithm which, e.g. based on the first fluorescence intensity, the second fluorescence intensity and the excitation wavelength of the light beam can derive parameters which may be further utilized to predict the composition, character and reactivity of DOM in the water under examination. The derived parameter may be a ratio of the first fluorescence intensity relative the second fluorescence intensity. The one or more databases can be connected to the server by a high bandwidth field area network (FAN) or wide area network (WAN), or can also be connected to the server through a wireless communication network.

The control circuitry 6 and each detector device 4, 5 may include, for example, one or more central processing units (CPUs), graphics processing units (GPUs) dedicated to performing calculations/transactions and/or other processing devices. The memory devices 8 can include one or more computer-readable media and can store information accessible by the control circuitry 6, including instructions/programs that can be executed by the control circuitry 6 so to operate the system 1.

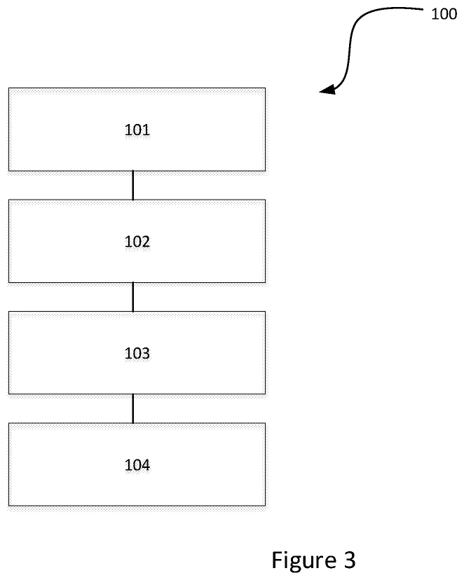

FIG. 3 illustrates a flowchart of a method 100 for determining the character, composition or reactivity of dissolved organic matter in water, the method 100 comprising the steps of exciting 101 a water under examination with an excitation emission having a wavelength in the range of 250-500 nm. Further, determining 102 a first fluorescence intensity emitted from said water by a first detector device measuring at a first emitting wavelength being in the range of 380-400 nm. Moreover, the method 100 comprises the step of determining 103 a second fluorescence intensity emitted from said water by a second detector device measuring at a second emitted wavelength being in the range of 490-580 nm. Furthermore, the method 100 comprises the step of predicting 104 at least one of the composition, character and reactivity of DOM of the water under examination based on said first and second fluorescence intensity.

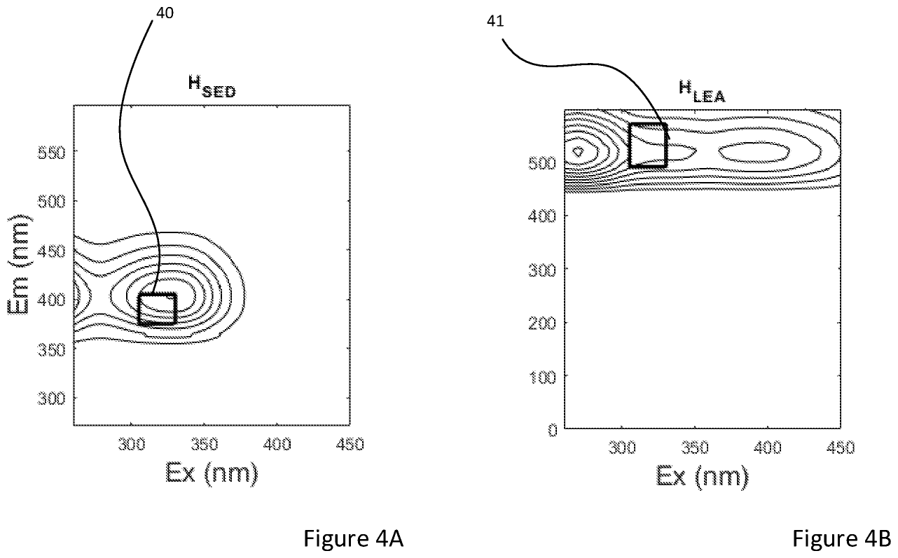

FIGS. 4-12 discloses a simulation and results of the system 1 and method 100 in accordance with aspects of the present disclosure. Thus, illustrating performance of the method 100 and system 1 as disclosed herein. The purpose of the FIGS. 4-11 is to further describe the disclosure as presented herein accompanied with advantages thereof. It should be noted that the Figures are based on embodiments for a disclosing purpose, however it is not limited to said embodiments and may be varied within the scope of the present disclosure.

FIGS. 4A-4B illustrates graphs showing the location of detector measuring windows 40, 41 in relation to DOM components that may be targeted by the control circuitry 6 (or algorithm thereof) of the present disclosure. Each graph 4A and 4B shows a contour plot representing the excitation and emission spectrum for a DOM component, with H_{LEA}=long-emission aromatic humics (in FIG. 4B) and H_{SED}=short-emission degraded humics (in FIG. 4A). The strongest signals occur where peaks are observed toward the middle of each cluster of contours. The measurement window 40 for the first detector device 4 may target H_{SED} while the measurement window 41 for the second detector device 5 may target H_{LEA}.

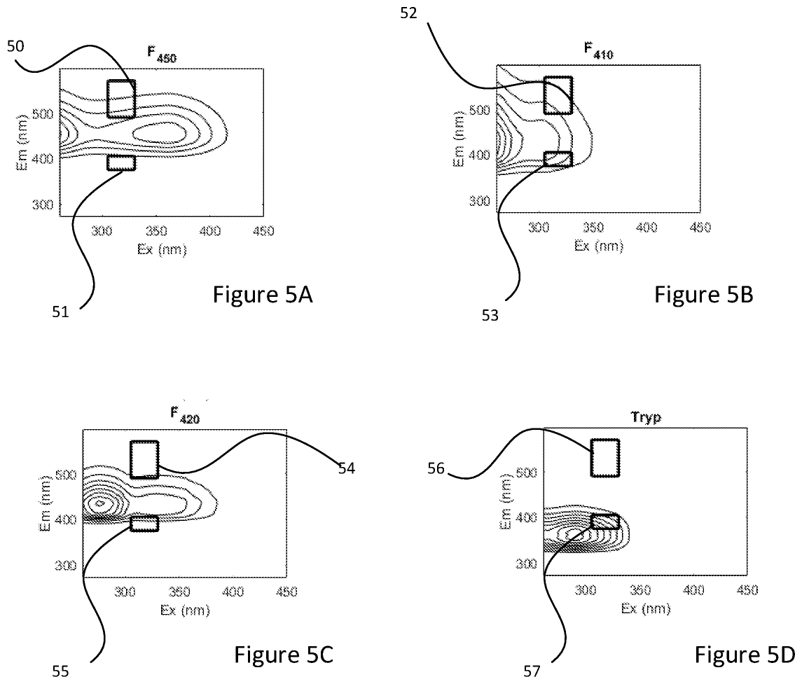

FIGS. 5A-5D illustrates graphs showing the location of measurement windows with reference numerals 50-57 belonging to the first and second detector devices 4, 5 in relation to non-target DOM components. The non-target components including F₄₁₀, F₄₂₀, F₄₅₀, and Tryp. Each of F₄₁₀, F₄₂₀, F₄₅₀, are “humic-like” components while Tryp is a “protein-like” component similar to tryptophan. The detector windows have been selected to minimise the potential for overlap with these non-target fluorophores.

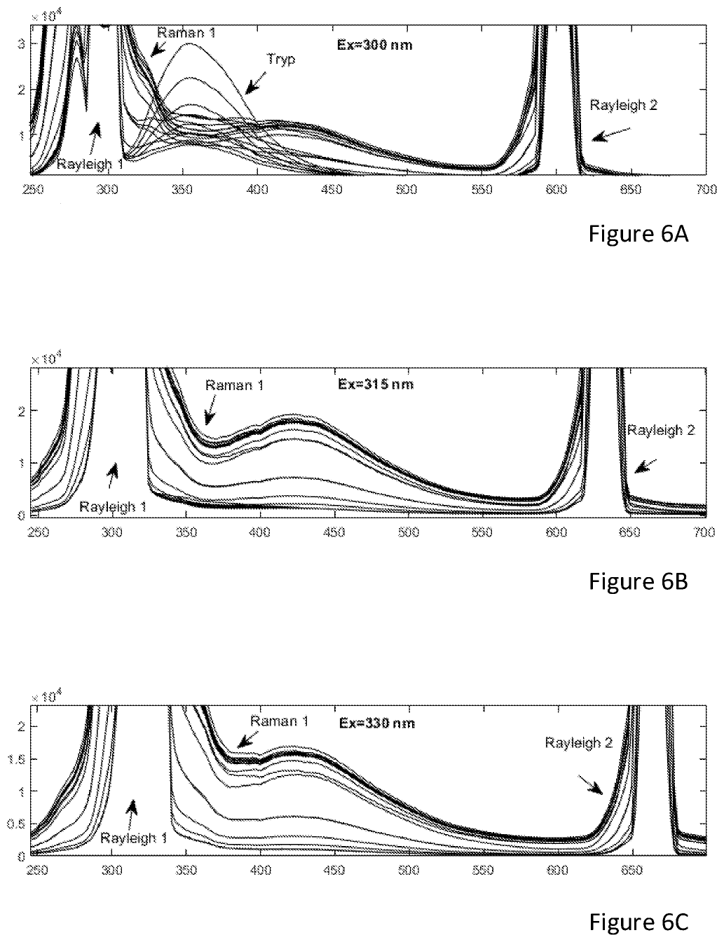

FIGS. 6A-6C illustrates graphs showing how the method 100 and system 1 of the present disclosure may be adapted within the disclosed wavelength ranges to avoid several types of interferences from non-target signals. The figures show scans for several water samples taken at excitation wavelengths, Ex=300, 315 and 330 nm. The samples have varying particle loads (turbidity) as seen by the variable width and height of Rayleigh scatter peaks in FIGS. 6A-6C. As turbidity increases the primary Rayleigh scatter bands centred at λ=2 Ex nm get broader and begin to overlap with the lower range of the H_{SED} detector positioned at 375-405 nm. At the same time, signals from the secondary Rayleigh scatter bands at λ=2 Ex nm get broader and begin to overlap with upper range of the measurement window for the H_{LEA} detector (Referred to in FIG. 4B). Additionally FIG. 6A shows interference in the form of protein-like (Tryp) fluorescence affecting the H_{SED} first detector window at relatively low excitation wavelengths (e.g. Ex=300 nm).

Based on the above, the optimal position for the measurement windows of the first device 4 detecting H_{SED} and second device 5 detecting H_{LEA} may therefore be varied in accordance with the scope of the present disclosure depending on the expected particle load in the water under examination 3 and whether particles will be removed prior to measurement, for example by using a prefilter device 16. Additionally, the optimal positions of the measurement windows depend on the cleanliness of the water and whether it contains measurable levels of fluorescence from proteins or amino acids—accordingly, said optimal positions of the measurement window are achievable by the method 100 and system 1 of the present disclosure. Protein-like fluorescence is common in water supplies that are impacted by wastewater, therefore the method 100 and system 1 of the present disclosure may also be appropriate for contaminated aquatic systems including wastewater treatment plants, compared to pristine aquatic systems including drinking water resources.

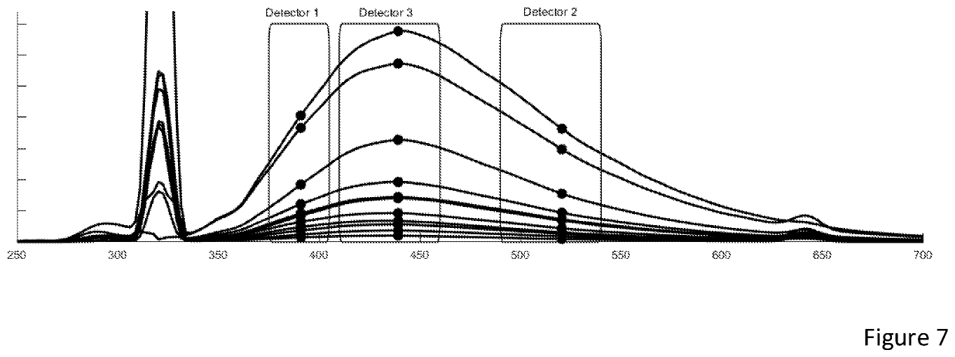

FIG. 7 illustrates a graph showing examples of the relationship between fluorescence signals, scatter peaks and detectors for ten samples collected in Alaskan rivers where with SUVA values ranged from 2.6-4.0 L/mg-m. FIG. 7 illustrates the relationships for the first detector 4 (denoted ‘Detector 1’ in FIG. 7), second detector 5 (denoted ‘Detector 2’ in FIG. 7) and the third detector 7 (denoted ‘Detector 3’ in FIG. 7) as illustrated in FIG. 2. However, in other aspects herein, one or more detectors may be replaced by a spectrometer covering the range of 375-580 nm. The first detector 4 is centred at 390 nm, the second detector 5 is placed at 520 nm, and the third detector 7 is placed at 440 nm. It would also be possible to replace the third detector 3 with several detectors covering the range of 405-490 nm to get a more precise representation of the full emission spectrum. By summing the measurements from three or more fluorescence detectors 4, 5, 7 it is possible to obtain a good estimate the total fluorescence emission for the sample (e.g. the water under examination 3) at selected excitation wavelengths. This in combination with an absorbance measurement obtained from additional optional UV detectors 14, 15 may enable estimation of the sample's apparent quantum yield (AQY). For aquatic samples containing fluorescent DOM from natural sources and insignificant levels of anthropogenic contamination, AQY values for water excited with light in the range of 305-335 nm are typically less than 1.8% whereas AQYs for dilute oil solutions are typically well above 10%.

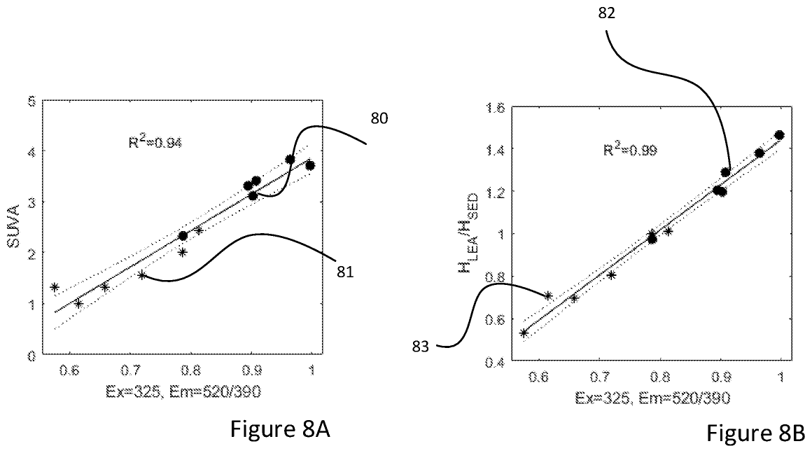

FIGS. 8A-8B illustrates graphs showing that the method 100 and system 1 of the present disclosure can be utilized to predict SUVA or to predict the ratio of aromatic to degraded DOM components (H_{LEA}/H_{SED}) and hence aromaticity and reactivity where FIGS. 8A-8B illustrates test result samples from two rivers in the Florida Everglades, USA. Reference numerals 80 and 82 illustrate a sample collected in the Harvey River and reference numerals 83 and 81 illustrate a sample collected in the Taylor River. By manipulating the equation for SUVA, the control circuitry 6 (shown in FIGS. 1 and 2) may be programmed to estimate DOC concentrations if supplied with a measurement of A254, measured either directly in the same device or in a separate device, or extrapolated from the absorbance measurement made at 305-335 excitation under the present disclosure. The method may also be used to predict nutrient ratios involving carbon (C), nitrogen (N), phosphorus (P), dissolved organic nitrogen (DON) or dissolved organic phosphorus (DOP), for example C:N, C:P, DOC:DON, or DOC:DOP.

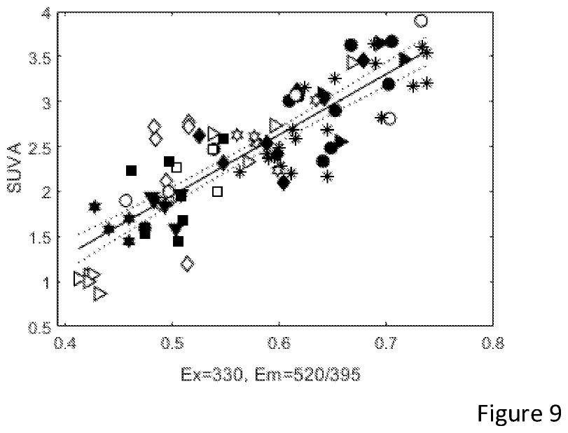

FIG. 9 is a graph showing that the method 100 and system 1 of the present disclosure may be utilized to predict SUVA and hence other water quality parameters that correlate with DOM reactivity, where FIG. 9 illustrates test samples from 15 rivers in the Canadian Yukon Valley subject to the method 100 and system 1 of the present disclosure.

The FIGS. 8A-9 illustrate that the method 100 and system 1 of the present disclosure may be utilized to determine the water quality in geographically variable locations having different climates, soil types, and vegetation types.

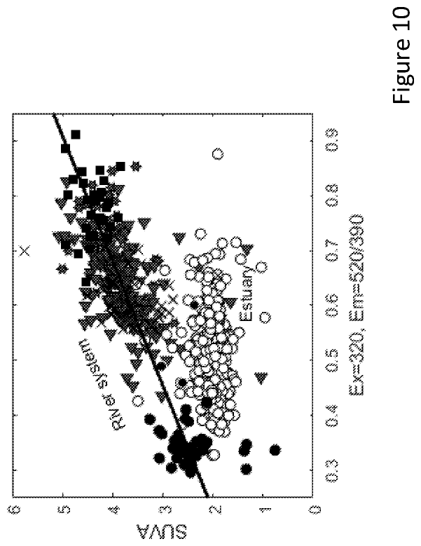

FIG. 10 is a graph demonstrating that the method 100 and system 1 of any aspect of the present disclosure may predict SUVA, where test samples from a freshwater system are disclosed. A good prediction of DOM reactivity is obtained for all samples collected in rivers and streams. In the estuary where the river signals are rapidly diluted by seawater, SUVA is expected to be relatively constant and the method can instead be used to detect hydrocarbons and other contaminants, for example, crude oils and their degradation products, which may produce anomalously high predictions according to the disclosed method 100 and system 1.

FIGS. 8, 9 and 10 show that the method 100 and system 1 of the present disclosure can be used to predict SUVA in samples from freshwater systems on at least two continents with good accuracy. It has been previously established that SUVA is a strong predictor of disinfection byproduct formation potential, irreversible and reversible membrane fouling potential, and optimal coagulant dose for advanced coagulation. Therefore, the method 100 and system 1 of the present disclosure may be incorporated into a decision support or control system for achieving a particular treatment outcome based on the method's prediction of SUVA. The control system could, for example, adjust chemical doses, flow rates and other operational conditions with the aim of (1) minimising the addition of chemicals during coagulation and minimising sludge production, (2) minimising irreversible fouling of membranes in order to decrease the frequency of deep chemical cleaning thereby extending membrane lifetimes, and (3) maintaining disinfection byproduct concentrations below regulated levels.

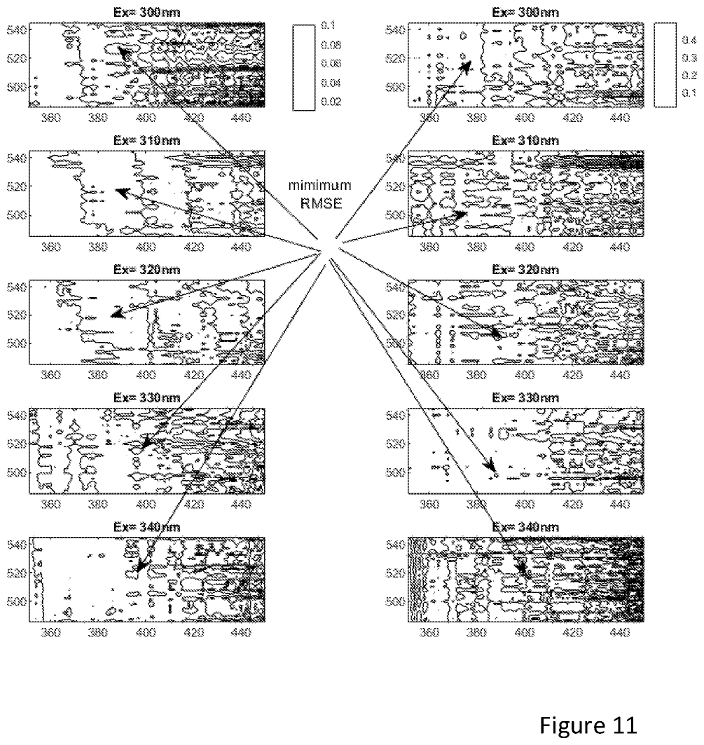

FIG. 11 is a graph showing the optimal measurement windows for predicting H_{LEA}/H_{SED} (left hand plots) and SUVA (right hand plots) using the method 100 and system 1 of the present disclosure applied to low turbidity samples from a dataset (also utilized in FIG. 8). The contours show error residuals (RMSE=root mean square error) for making a prediction by linear regression according to the method 100 and system 1 of the present disclosure. FIG. 11 illustrates that the method and system of the present disclosure is suitable for predicting H_{LEA}/H_{SED} and SUVA, this is evident by the low RMSE illustrated at the parts of the plots being within the ranges disclosed (Ex/Em ranges) by the present disclosure.

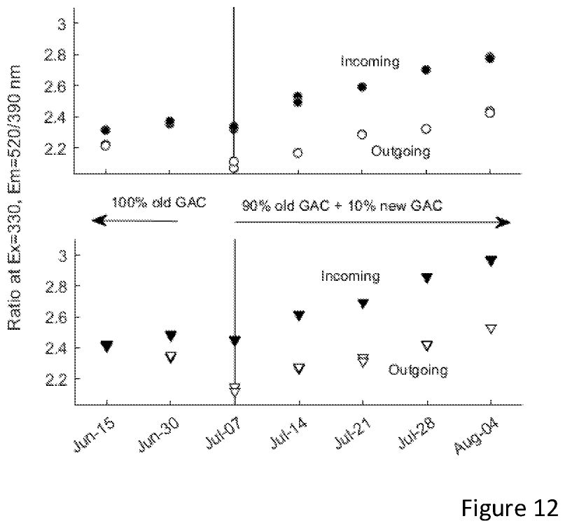

FIG. 12 is a graph showing that the method 100 and system 1 of the present disclosure may be utilized to quantify changes in DOM composition caused by adsorption onto granular activated carbon (GAC) filters. The degree of GAC saturation is indicated by the difference between the ratio predicted by the method for water entering the GAC (incoming) compared to water leaving the GAC (outgoing). New GAC has much higher adsorption capacity than does old (>6 months) GAC and therefore has a larger difference between incoming and outgoing water. The method 100 and system 1 of the present disclosure could therefore be incorporated into a decision support system for determining the appropriate time to recharge or replace GAC media, or a control system that determines the operational conditions for a GAC filter.

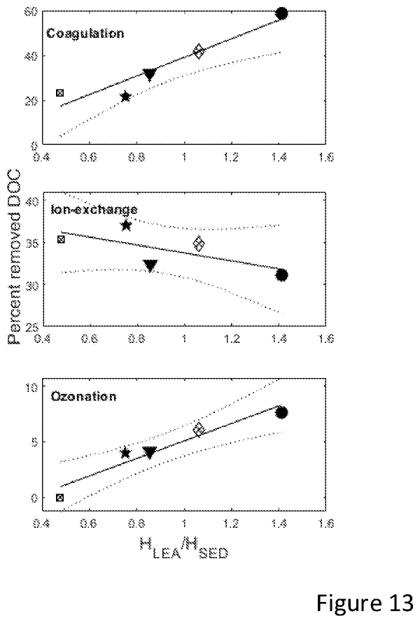

FIG. 13 is a graph showing that the method 100 and system 1 of the present disclosure may be utilized to predict drinking water treatability (i.e. DOC removal) at various stages of water treatment. The figure shows in three graphs that for five water sources treated by coagulation, ion-exchange, and ozonation, fluorescence composition was linearly correlated to DOC removal following treatment.

1. A system for determining at least one of character, composition and reactivity of dissolved organic matter, DOM in water comprising:
an ultraviolet, UV light source configured to excite a water under examination with a light beam having an excitation wavelength in a range of 250-500 nm;
a first detector device configured to determine a first fluorescence intensity emitted from said water under examination, the first detector device being arranged to measure at a first emitting wavelength being in a range of 375-405 nm;
a second detector device configured to determine a second fluorescence intensity emitted from said water under examination, the second detector device being arranged to measure at a second emitting wavelength being in a range of 490-580 nm;
wherein the system further comprises control circuitry configured to:
predict at least one of the composition, character and reactivity of DOM in the water under examination based on said first and second fluorescence intensity.

2. The system according to claim 1, wherein the first and the second detector devices are integrated, forming a common detector device being arranged to measure at said first and second emitting wavelength.

3. The system according to claim 1, wherein the excitation wavelength is in a range of 305-335 nm, preferably 310-330 nm, more preferably 313-325 nm.

4. The system according to claim 1, wherein the first emitting wavelength is in a range of 380-400 nm, preferably, 385-395 nm.

5. The system according to claim 1, wherein the second emitted wavelength is in a range of 500-570 nm, preferably 510-540 nm.

6. The system according to claim 1, wherein the control circuitry is configured to predict based on a ratio of the first fluorescence intensity relative the second fluorescence intensity.

7. The system according to claim 1, further comprising a third detector device configured to determine at a third fluorescence intensity emitted from said water under examination, the third detector device being arranged to measure at a third emitted wavelength being in a range of 410-460 nm, said control circuitry being configured to further base the prediction on said third fluorescence intensity.

8. The system according to claim 1, further comprising:
a first ultraviolet detector device configured to monitor an intensity of said ultraviolet light source;
a second ultraviolet detector device configured to measure an amount of light transmitted through said water under examination; and
wherein said control circuitry is further configured to apply a correction factor to said prediction, said correction factor being based on the measured amount of light transmitted through said water under examination and the monitored intensity of said ultraviolet light source.

9. The system according to claim 1, further comprising a pre-filter device configured to exclude particles equal to or greater than 0.2 □m from the water under examination prior to determining fluorescence intensity by means of the first and the second detector device.

10. The system according to claim 1, wherein the UV light source is a first light source having an excitation wavelength of 305-335 nm, wherein the system further comprises a second UV light source configured to excite the water under examination with another light beam, the another light beam having an excitation wavelength in a range of 250-500 nm, wherein the first detector is arranged to detect fluorescence emissions due to the first light source and the second detector is arranged to detect fluorescence emissions due to the second light source.

11. A method for determining character, composition or reactivity of dissolved organic matter, DOM in water, the method comprising the steps of:
exciting a water under examination with an excitation emission having a wavelength in a range of 250-500 nm;
determining a first fluorescence intensity emitted from said water by a first detector device measuring at a first emitting wavelength being in a range of 375-405 nm;
determining a second fluorescence intensity emitted from said water by a second detector device measuring at a second emitted wavelength being in a range of 490-580 nm
predicting at least one of the composition, character, and reactivity of DOM of the water under examination based on said first and second fluorescence intensity.

12. A computer-readable storage medium storing one or more programs configured to be executed by one or more control circuitry of a system, the one or more programs including instructions for performing the method of claim 11.
